(19) United States
(12) Patent Application Publication
(10) Pub. No.: US 2017/0035525 A1
(43) Pub. Date: Feb. 9, 2017

(54) Neurosurgical Alignment and Treatment Devices

(71) Applicant: ADVENTIST HEALTH SYSTEM/SUNBELT, INC., Altamonte Springs, FL (US)

(72) Inventor: James Edmund Baumgartner, Winter Park, FL (US)

(21) Appl. No.: 15/225,549

(22) Filed: Aug. 1, 2016

Related U.S. Application Data
(60) Provisional application No. 62/200,297, filed on Aug. 3, 2015.

(51) Int. Cl. A61B 90/10; A61B 18/20

(52) U.S. Cl. CPC A61B 90/10; A61B 18/20; A61B 2018/00446

(57) ABSTRACT

The present disclosure provides systems and methods for performing surgery on a patient's skull or brain. A mounting and stabilization device is provided for frameless, trajectory-based laser ablation surgery. The device employs a simple aiming mechanic and can be used to treat patients such as pediatric patients that cannot be treated with other devices and methods.

### RELATED APPLICATION

This application claims the benefit of priority of U.S. Provisional Application No. 62/200,297, entitled “Neurosurgical Alignment And Treatment Devices” and filed Aug. 3, 2015, the content of which is hereby incorporated by reference in its entirety.

### BACKGROUND

The present disclosure relates to surgical devices and methods for neurosurgical alignment and treatment.

Existing systems and methods for neurosurgery include the use of a stereo-tactic frame to restrict the movements of the patient and guide instruments to appropriate surgical structures. Initial setup of such frames, however, can be time-consuming, and use of the frame may be cumbersome as it can block the surgeon's line-of-sight or the motion of surgical instruments during the operation.

The use of trajectory-based neurosurgical methods has increased as the accuracy and reliability of such systems has improved. Also known as frameless guiding methods, they can be adapted to a range of techniques including brain biopsy, tumor resection, and radiation treatment without the need for a full frame to stabilize the head.

Laser ablation surgery is a treatment method that has been used in the treatment of deep-seated brain lesions, metastatic tumors, and primary brain tumors. In addition, surgeons can use the technology to ablate seizure foci in epilepsy patients. In laser ablation surgery, a burr hole is created in the patient's skull to provide visualization of surface vessels. In previous systems, targeting was accomplished by using an external frame (stereotactic frame) or a skull-mounted tripod. Both of these systems require significant setup time in the operating theater, and they typically limit the surgeon's freedom to align the proper trajectory due to their fixed nature. In many systems, a large titanium bolt is driven into the patient's skull to fix the location of the laser ablation device. Unfortunately, such a large bolt is unsuitable for younger patients (i.e., patients under 5 years old) due to their softer skulls.

### SUMMARY

The present disclosure describes several devices and methods for frameless, trajectory-based laser ablation surgery. According to certain embodiments, a surgery system is provided. The device can be used to accurately and precisely guide surgical instruments to treat and/or provide diagnostic procedures on the brain or other associated structures. The device allows precise positioning of instruments for resection, biopsy, and/or ablation of tissues and for positioning of probes or other instruments (e.g., electrodes or other diagnostic tools).

In accordance with various embodiments, a neurosurgery system includes a frameless guide and a ball-stem adapter. The ball-stem adapter has a distal end that is adapted to attach to the frameless guide. The ball-stem adapter also has a proximal end that is adapted to be coupled to a laser ablation device.

In accordance with various embodiments, a method of performing a surgical procedure on a skull or brain is provided. The method includes attaching a distal end of a ball-stem adapter to a frameless guide and mounting the frameless guide to a skull of a patient. The method also includes coupling a laser ablation device to the proximal end of the ball-stem adapter and performing a laser ablation operation.

In accordance with various embodiments, a surgical mounting device is provided. The device includes a frameless guide and a ball-stem adapter. The ball-stem adapter has a distal end that is adapted to attach to the frameless guide and a proximal end that is adapted to be coupled to a surgical instrument. The surgical mounting device includes a plurality of MRI-compatible fiducial markers.

### BRIEF DESCRIPTION OF THE DRAWINGS

FIG. 1 illustrates a system for frameless, trajectory-based laser ablation surgery according to various embodiments.

FIG. 2 illustrates a perspective view of a mounting and stabilization device for frameless, trajectory-based laser ablation surgery according to various embodiments.

FIGS. 3A and 3B illustrate side views of a mounting and stabilization device for frameless, trajectory-based laser ablation surgery according to various embodiments.

FIG. 4 illustrates a top view of a mounting and stabilization device for frameless, trajectory-based laser ablation surgery according to various embodiments.

FIG. 5 illustrates a method of performing a surgical procedure on a skull or brain according to various embodiments.

FIG. 6 illustrates a mounting and stabilization device attached to the skull of a patient according to various embodiments.

### DESCRIPTION OF CERTAIN EXEMPLARY EMBODIMENTS

Reference will now be made in detail to certain exemplary embodiments according to the present disclosure, certain examples of which are illustrated in the accompanying drawings. Wherever possible, the same reference numbers will be used throughout the drawings to refer to the same or like parts.

In this application, the use of the singular includes the plural unless specifically stated otherwise. In this application, the use of “or” means “and/or” unless stated otherwise. Furthermore, the use of the term “including”, as well as other forms such as “included” and “includes”, is not limiting.

Use of the word “distal” is intended to indicate portions of an object nearest the patient while the word “proximal” is intended to indicate portions of an object furthest from the patient. Any range described herein will be understood to include the endpoints and all values between the endpoints.

The section headings used herein are for organizational purposes only and are not to be construed as limiting the subject matter described. All documents, or portions of documents, cited in this application including but not limited to patents, patent applications, articles, books, and treatises are hereby expressly incorporated by reference in their entirety for any purpose.

As discussed above, laser ablation surgery is useful in treating maladies such as primary and metastatic tumors and epilepsy. Current surgical methods provide inadequate angular and positional flexibility to the surgeon in terms of aiming the trajectory of the device. In addition, present methods of securing the device to the skull of the patient (i.e., a large titanium bolt) may have shortcomings for patients with softer and smaller skulls, including pediatric patients under five years of age.

Herein, we present methods and devices for frameless, trajectory-based laser ablation surgery. According to certain embodiments, a surgery system is provided. The device can be used to accurately and precisely guide surgical instruments to treat and/or provide diagnostic procedures on the brain or other associated structures. The device allows precise positioning of instruments for resection, biopsy, and/or ablation of tissues and for positioning of probes or other instruments (e.g., electrodes or other diagnostic tools).

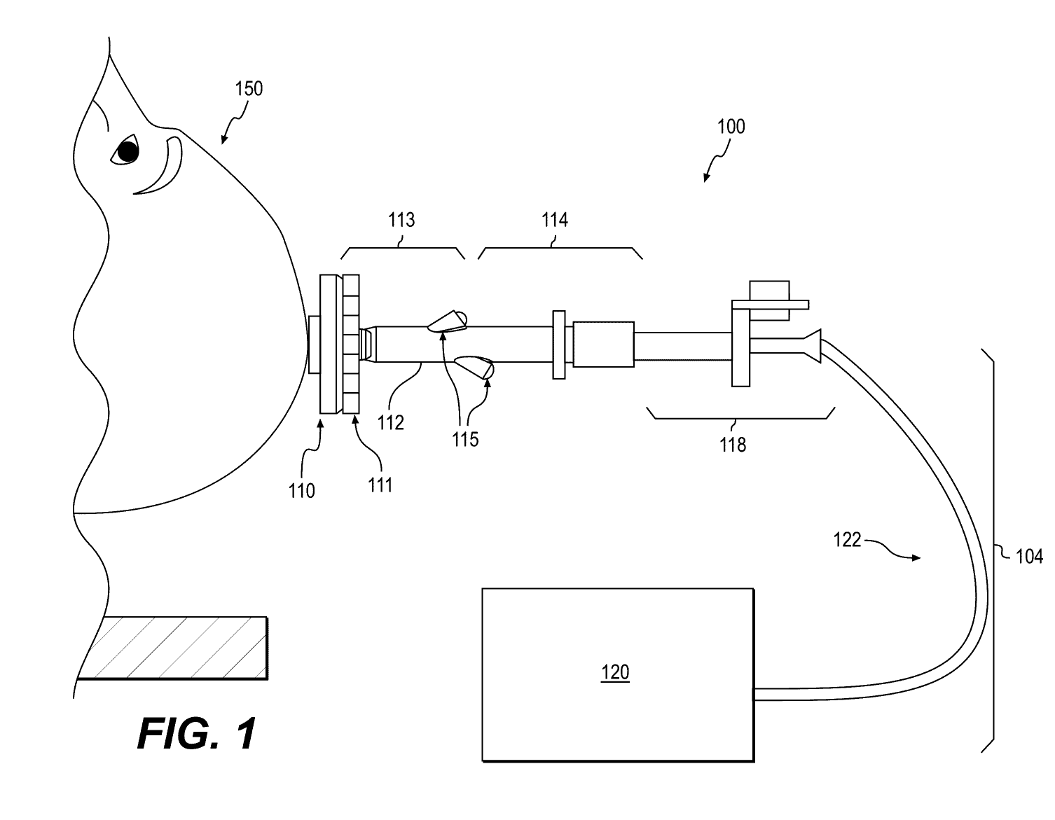

According to certain embodiments, a laser ablation surgery system is provided. With reference to FIG. 1, a neurosurgery system 100 may include a mounting system 102 and a laser ablation system 104. The mounting system 102 can include a frameless guide 110, a retainer ring 111, and a ball-stem adapter 112. The frameless guide 110 may be fixedly and removably attached over a site (e.g., a burr hole or other opening) in a patient's skull 150. A distal portion 113 of a ball-stem adapter 112 may be held fast to the frameless guide 110 using the retainer ring 111. The proximal portion 114 of the ball-stem adapter may include an end adapter 116. The laser ablation system 104 can include a laser ablation probe 118 and laser ablation controls 120. The laser ablation probe 118 can be attached to the end adapter 116. The laser ablation probe 118 is connected to the laser ablation controls 120. The laser ablation probe 118 extends through the ball-stem adapter mounting system 102 and into the patient's brain through the surgical hole. Because the mounting system 102 can have a relatively low profile and is relatively easy to attach to the skull 150, the neurosurgery system 100 can be used for virtually any desired skull entry point or biopsy target. Because laser ablation (accomplished after the mounting system 102 has been secured to a skull 150 in an operating room) is often performed within the lumen of an MRI machine, the low profile of the mounting system 102 can considerably increase entry point options for the operating surgeon. The low profile of the mounting system 102 also can improve ease of use for the surgical team in terms of accessing the laser and targeting machinery.

The neurosurgery system 100 can be used to deliver laser light directly to portions of the brain or other structures. According to various embodiments, the laser light may be used to heat or ablate soft tissue in a localized fashion such that only a limited region of the brain is affected. Localized tissue heating and ablation are desirable methods to treat diseases focused in well-defined portions of the brain including, but not limited to, tumors or lesions because impacts can be limited to only the targeted region while leaving other regions of the brain intact. Moreover, the neurosurgery system 100 can be used to deliver other instruments such as electrodes for mapping, DBS probes, and biopsy needles directly to portions of the brain or other structures.

In accordance with various embodiments, the laser ablation system 104 is comprised of a laser ablation probe 118 and laser ablation controls 120. The laser ablation probe 118 and laser ablation controls 120 may be connected by cables 122. In various embodiments, the laser ablation controls 120 can provide the laser ablation probe 118 with laser light, cooling fluid, and/or positional control. The laser ablation probe 118 may be side-firing or forward-firing. Exemplary laser ablation systems 104 are the NEUROBLATE® SYSTEM (MONTERIS MEDICAL, Plymouth, Minn.) and the VISUALASE® device (Medtronics, Minneapolis, Minn.).

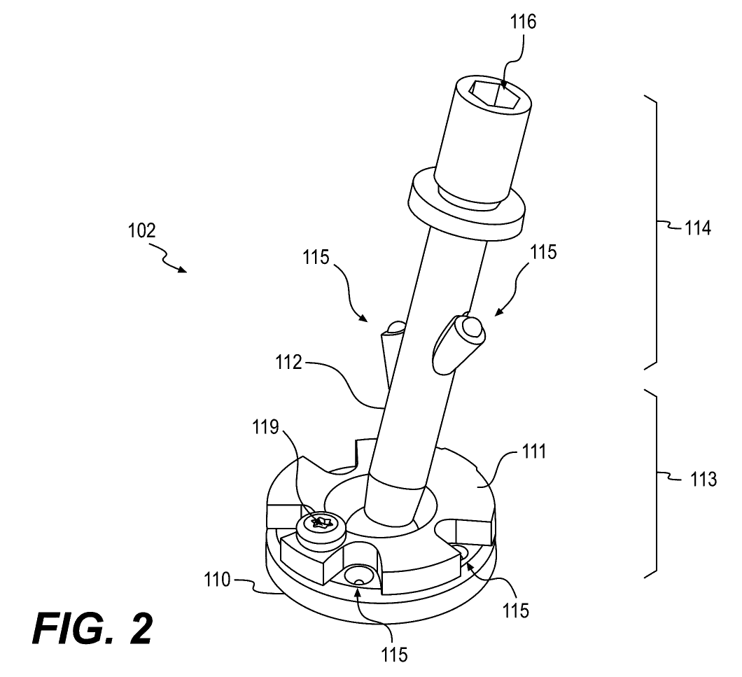

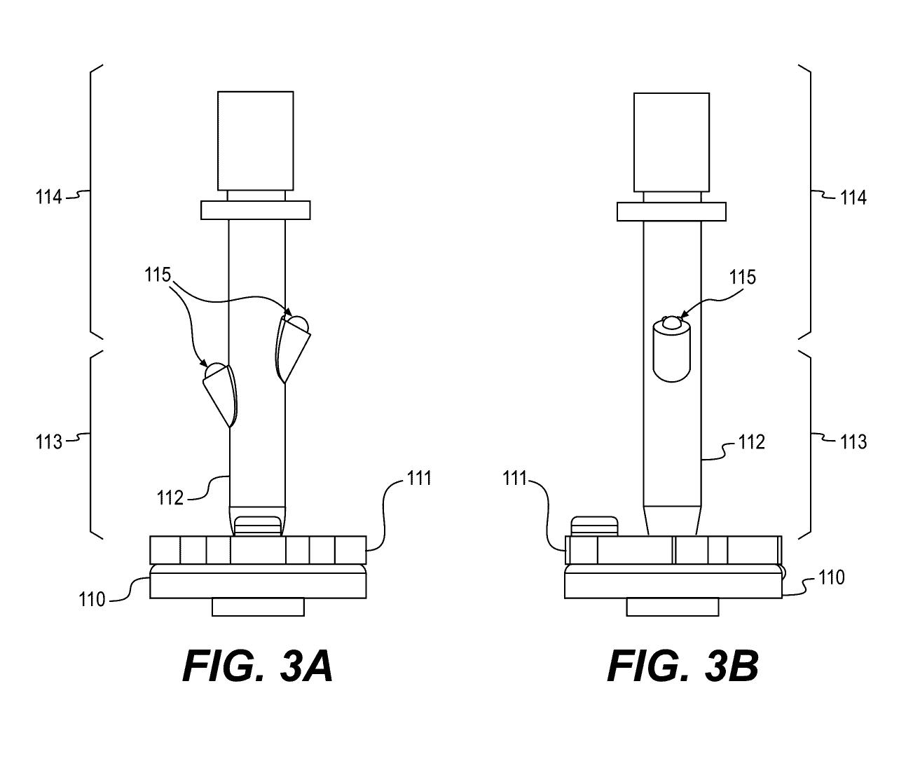

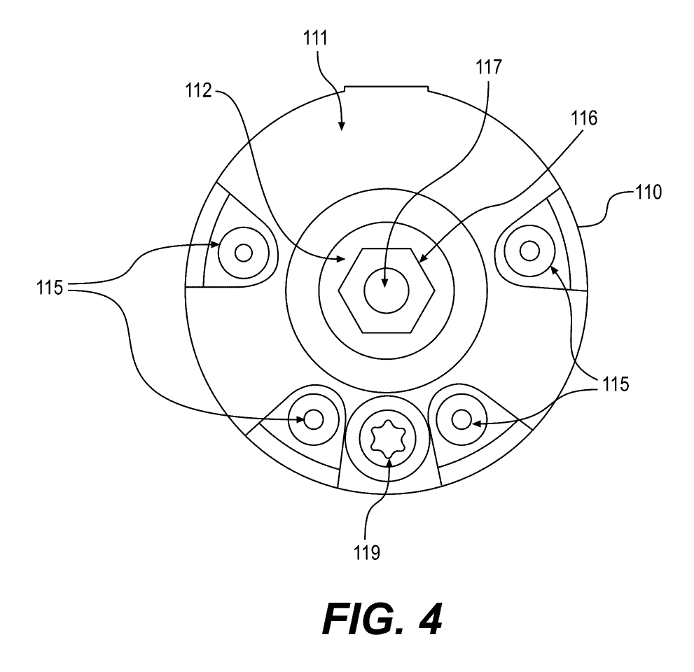

With reference to FIGS. 2, 3A, 3B, and 4, perspective, side, and end views of a mounting system 102 are illustrated. The mounting system 102 can generally include a frameless guide 110, a retainer ring 111, and a ball-stem adapter 112. The mounting system 102 can reliably and securely fasten the laser ablation probe 118 to the skull 150 of a patient such that relative movements between the probe 118 and the patient's skull 150 are not possible.

The frameless guide 110 can be made of any suitable material that is non-reactive in an MRI environment (e.g., MRI compatible polymers such as polyether ether ketone [PEEK] or polyetherimide [PEI; also known as ULTEM®]). In an exemplary embodiment, the frameless guide can be a base plate with a plurality of evenly spaced, counter-bored through-holes. The base plate may have any form factor including, but not limited to, a flat or round body. In embodiments where the base plate is flat, it may have any circumferential shape including, but not limited to, circle, square, hexagon, oval, or polygon and may be symmetric or asymmetric. In accordance with various embodiments, the frameless guide 110 may be placed over a burr hole drilled into the patient's skull 150 to allow access to the brain and visualization of surface vessels. The frameless guide 110 may be secured to the patient's skull 150 using small screws. In a preferred embodiment, the screws can be M2 metric screws or smaller. In accordance with certain embodiments, the frameless guide 110 can include a latch or tongue that is configured to engage with a hole in the retainer ring. The latch or tongue may provide a hinging action in the way that the retainer ring 111 is attached to the frameless guide 110 to enable fast disengagement and reseating of the retainer ring 111 to the frameless guide 110. In certain embodiments, the frameless guide 110 may include a locking screw hole that is threaded to accept a locking screw 119.

The center of the frameless guide may contain a hole to provide access into the burr hole of the patient's skull 150. In various embodiments, the wall of the through-hole may be straight or it may contained chamfered, sloped, curved, cup-like, or socket-like sections to receive and support the ball-stem adapter 112. In an exemplary embodiment, the frameless guide 110 may be the frameless guide component of the IGS or FGS FRAMELESS GUIDING SYSTEM (STRYKER NAVIGATION, Kalamazoo, Mich.).

The retainer ring 111 can be made of any suitable material that is non-reactive in an MRI environment (e.g., MRI compatible polymers such as polyether ether ketone [PEEK] or polyetherimide [PEI; also known as Ultem®]). In accordance with various embodiments, the retainer ring 111 may contain a hole that can operatively engage with a latch or tongue on the frameless guide 110. In an exemplary embodiment, the retainer ring contains a through-hole that allows the passage of a locking screw 119. The locking screw through-hole may be disposed on an opposite portion of the retainer ring 111 from the hole for the latch or tongue. Locking screw 119 can engage with the threaded locking screw hole in the frameless guide 110 and can create firm but reversible contact between the retainer ring 111 and the frameless guide 110. The locking screw 119 and latch hole enable quick and reliable positioning of the ball-stem adapter 112 at appropriate angles. In accordance with various embodiments, the retainer ring 111 may have one or more cutouts that reveal portions of the top surface of the frameless guide 110 when the two parts are mated.

The ball-stem adapter 112 can be made of any suitable material that is non-reactive in an MRI environment (e.g., MRI compatible polymers such as polyether ether ketone [PEEK] or polyetherimide [PEI; also known as Ultem®]). The ball-stem adapter 112 can help to properly set the trajectory of the laser ablation probe 118 as it enters the skull 150. The ball-stem adapter may contain a through-hole 117 that passes through the center of the adapter between the proximal end and the distal end. In various embodiments, the internal diameter of the through-hole 117 of the ball-stem adapter 112 may be substantially constant throughout its entire length or may vary gradually or discretely along some segments of the length. The internal diameter of the through-hole 117 may be chosen to be any size that satisfies application-specific requirements. In accordance with various embodiments, the distal portion 113 of the ball-stem adapter 112 may comprise a substantially spherical or ellipsoidal shape. The shape of the distal end 113 of the ball-stem adapter 112 may be mirrored in the shape of the through-hole in the frameless guide 110. A spherical or ellipsoidal shape of the distal end 113 of the ball-stem adapter 112 can enable the adapter to be rotated about its longitudinal axis to a position that is most convenient for use by the physician. In addition, the ball-stem adapter 112 may be tilted with respect to the normal defined by the frameless guide 110 to access trajectories that are away from the normal axis. In this way, the ball-stem adapter 112 maintains two degrees of rotational freedom, and multiple trajectories may be accessed using a single burr hole. In accordance with various embodiments, the proximal portion 114 of the ball-stem adapter 112 may comprise an end adapter 116 that is configured to allow mounting of the laser ablation probe 118.

In various embodiments, the internal diameter of an end adapter 116 may be circular, hexagonal, octagonal, square, polygonal, or any other suitable shape required by the application or by the mounting mechanism of a laser ablation probe 118 or other suitable system. The end adapter 116 may comprise one or more set screws, knob screws, or any other suitable element to mount a laser ablation probe 118 to the ball-stem adapter 112. According to various embodiments, the proximal portion 114 of the ball-stem adapter 112 may comprise a ring or collar around its exterior diameter. This ring or collar may be used to enable mounting of the laser ablation probe 118 or for ease in grasping the ball-stem adapter 112.

In various embodiments, the frameless guide 110 and/or the ball-stem adapter 112 may include one or more fiducial markers 115. The fiducial markers 115 allow a user to register the position of the frameless guide 110 and/or the ball-stem adapter 112 in three-dimensional space over time and/or with previously-obtained imagery. The plurality of fiducial markers 115 may be imaged using one or more of a wide variety of imaging modalities including, but not limited to, magnetic resonance imaging, positron emission tomography, and/or x-ray imaging. In a preferred embodiment, the plurality of fiducial markers 115 are made from a non-paramagnetic material such as gold. The fiducial markers 115 may be attached to the frameless guide 110 and/or ball-stem adapter 112 using a variety of methods including, but not limited to, adhesive mounting, friction fit, retainer rings, welding, sintering, or vapor deposition. In accordance with various embodiments, the fiducial markers can be 2 mm spheres, and the distance between the markers may be about 10 mm. In the embodiment depicted in FIG. 2, the retainer ring 111 has several cutouts that allow access to the fiducial markers 115 mounted on the frameless guide 110.

In accordance with various embodiments, a physician may use the plurality of fiducial markers 115 to register the location of the mounting system 102 and, hence, the patient's skull 150 during a surgical operation. The location of the fiducial markers 115 may be obtained using, for example, magnetic resonance imaging. As an example, the plurality of fiducial markers 115 can be identified on an MRI scan that is obtained in the MRI suite after the mounting device 102 has been secured to a patient's skull 150. The fiducial markers 115 can then be registered to the navigation system used in the operating room. With this registration, the location of the ball-stem adapter 112 on the skull 150 and the trajectory defined by the lumen 117 of the ball-stem adapter 112 can be identified and shown on a navigation screen. The depth to a potential target can be measured, and the path chosen for the laser fiber can be examined for proximity to critical structures (such as blood vessels or anatomically delicate structures) before the fiber is advanced intracranially. With this approach, adjustments to the trajectory can be made before passing the laser fiber to avoid injury to critical structures. In addition, multiple trajectories can be utilized through a single opening by reorienting the trajectory of the ball-stem adapter 112 and re-registering to the navigation system. The fiducial markers 115 can provide advantages in terms of the time required to register the device and calculate a trajectory as compared to existing products. In addition, the ease and speed with which multiple trajectories can be calculated and executed are far superior to existing devices. In accordance with some embodiments, the total time (excluding scanning) required to calculate, evaluate and utilize a single planned trajectory, post intra-op MRI can be under thirty minutes whereas existing frame-based devices often require over 90 minutes to complete the same task. When setting up multiple trajectories, the additional time required with certain embodiments of this disclosure can be less than 20 minutes and would not require creation of a second entry point. Conversely, addressing multiple brain sites with a frame-based system can add an additional 45 minutes to one hour to the procedure.

By storing MRI images in a computer, the location of the fiducial markers 115 can be tracked over the time of the operation. In addition, the location of the fiducial markers 115 may be registered to previously acquired images of the patient's brain. In a preferred embodiment, the path from the exterior of the patient's skull 150 to the targeted, diseased portion of the brain is optimized using the location of the fiducial markers 115.

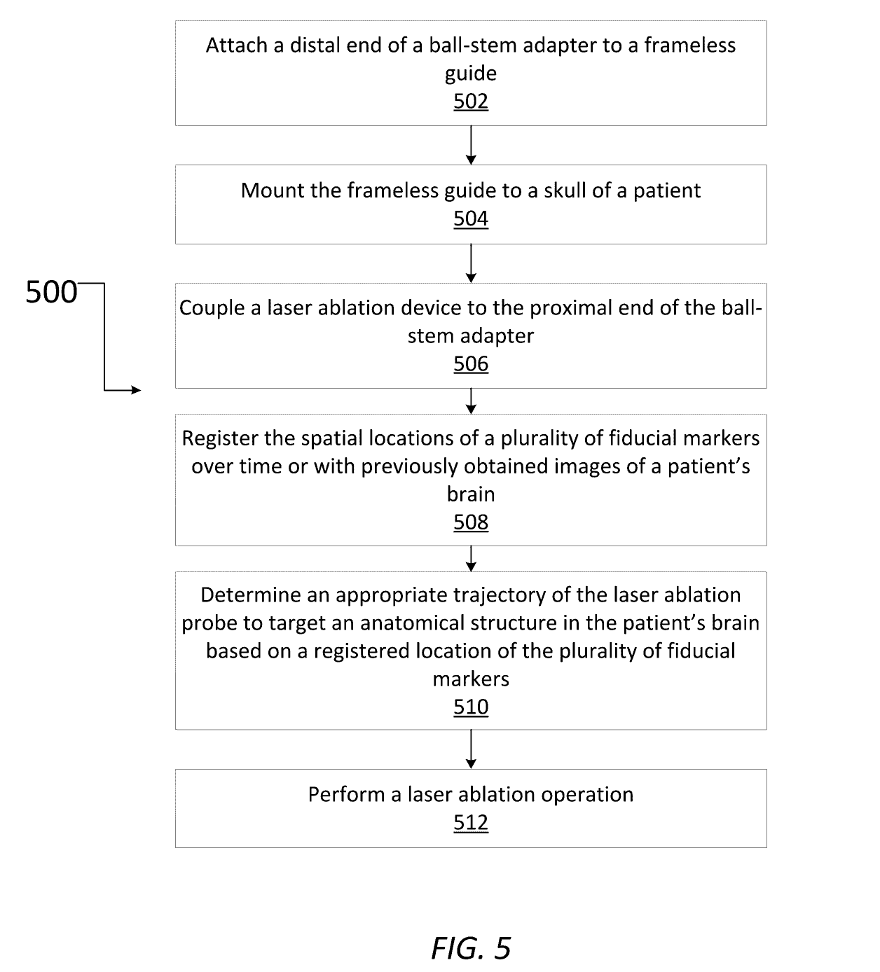

With reference to FIG. 5, a method 500 of performing a surgical procedure on a skull or brain is illustrated. The method 500 includes a step of attaching 502 a distal end of a ball-stem adapter to a frameless guide. The method 500 includes a step of mounting 504 the frameless guide to a skull of a patient. The method 500 includes a step of coupling 506 a laser ablation device to the proximal end of the ball-stem adapter. The method 500 includes a step of registering 508 the spatial locations of a plurality of fiducial markers over time or with previously obtained images of a patient's brain. The method 500 includes a step of determining 510 an appropriate trajectory of the laser ablation probe to target an anatomical structure in the patient's brain based on a registered location of the plurality of fiducial markers. The method 500 includes a step of performing 512 a laser ablation operation.

The step of attaching 502 a distal end of a ball-stem adapter to a frameless guide can include, for example but not limited to, retaining a distal end 113 of a ball-stem adapter 112 using a retainer ring 111 and a frameless guide 110 as described above with reference to FIG. 2.

The step of mounting 504 the frameless guide to a skull of a patient can include, for example but not limited to, using surgical screws to mount a frameless guide 110 to a skull 150 as described above with reference to FIG. 2.

The step of coupling 506 a laser ablation device to the proximal end of the ball-stem adapter can include, for example but not limited to, affixing a laser ablation probe 118 to an end adapter 116 at a proximal end 114 of a ball-stem adapter 112 as described above with reference to FIGS. 1 and 2.

The step of registering 508 the spatial locations of a plurality of fiducial markers over time or with previously obtained images of a patient's brain can include, for example but not limited to, imaging a plurality of fiducial markers 115 using magnetic resonance imaging and tracking the locations between subsequent image acquisitions or comparing the locations to previously obtained imagery of the patient's brain 150 as described above with reference to FIG. 2.

The step of determining 510 an appropriate trajectory of the laser ablation probe to target an anatomical structure in the patient's brain based on a registered location of the plurality of fiducial markers can include, for example but not limited to, using magnetic resonance imaging to image the laser ablation probe 118 as it advances into the patient's skull 150 and comparing that location information with trajectory information acquired from a plurality of fiducial markers 115 and the known location of diseased areas of the brain determined by previously acquired images as described above with reference to FIG. 2

The step of performing 512 a laser ablation operation can include, for example but not limited to, selectively activating a laser ablation probe 118 when it is positioned proximal to diseased regions of a patient's brain as described above with reference to FIG. 1.

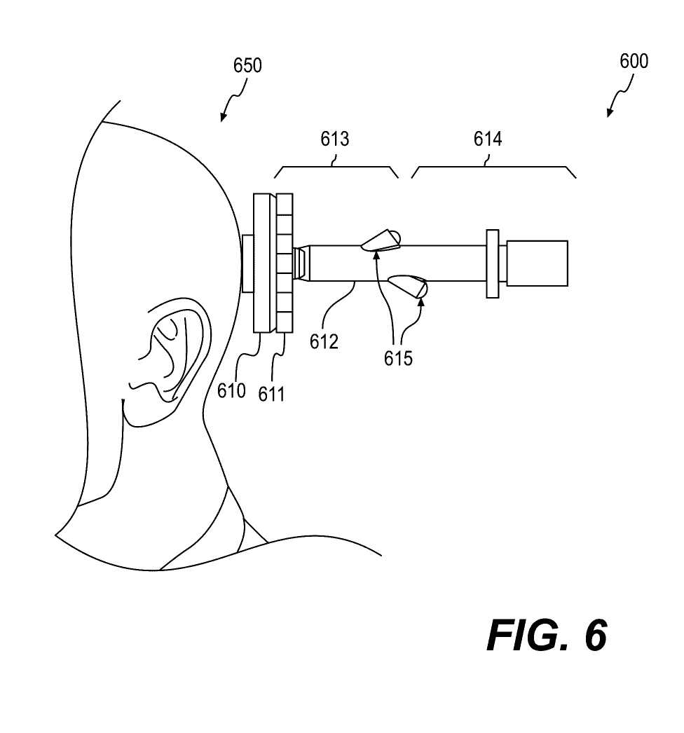

With reference to FIG. 6, a mounting system 600 can comprise a frameless guide 610 and a ball-stem adapter 612. The ball-stem adapter 612 can include a distal end 613 and a proximal end 614. A plurality of fiducial markers 615 may be located on the mounting system 600. In accordance with various embodiments, the frameless guide 610 of the mounting system 600 can be attached to a patient's skull 650. The mounting system 600 may be adapted to reliably and securely fasten a surgical instrument to a patient's skull 650. For example, a surgical instrument might include, but is not limited to, a laser ablation system, stimulation electrodes, biopsy needles, or sensor electrodes. In accordance with various embodiments, the frameless guide 610 of the mounting system 600 may be attached to a skull 650 of a patient near the patient's occipital lobe to provide better access to this brain region. In an alternate embodiment, the frameless guide 610 may be attached to a skull 650 of a patient near the patient's parietal lobe to provide better access to this brain region.

The frameless guide 610 can be made of any suitable material that is non-reactive in an MRI environment (e.g., MRI compatible polymers such as polyether ether ketone [PEEK] or polyetherimide [PEI; also known as Ultem®]). In an exemplary embodiment, the frameless guide 610 can be a base plate with a plurality of evenly spaced, counter-bored through-holes. The base plate may have any form factor including, but not limited to, a flat or round body. In embodiments where the base plate is flat, it may have any circumferential shape including, but not limited to, circle, square, hexagon, oval, or polygon and may be symmetric or asymmetric. In accordance with various embodiments, the frameless guide 610 may be placed over a burr hole drilled into the patient's skull 650 to allow access to the brain and visualization of surface vessels. The frameless guide 610 may be secured to the patient's skull 650 using small screws. In a preferred embodiment, the screws can be M2 metric screws or smaller. In accordance with certain embodiments, the frameless guide 610 can include a latch or tongue that is configured to engage with a hole in the retainer ring. The latch or tongue may provide a hinging action in the way that the retainer ring 611 is attached to the frameless guide 610 to enable fast disengagement and reseating of the retainer ring 611 to the frameless guide 610. In certain embodiments, the frameless guide 610 may include a locking screw hole that is threaded to accept a locking screw.

The center of the frameless guide 610 may contain a hole to provide access into the burr hole of the patient's skull 650. In various embodiments, the wall of the through-hole may be straight or it may contained chamfered, sloped, curved, cup-like, or socket-like sections to receive and support the ball-stem adapter 612. In an exemplary embodiment, the frameless guide 610 may be the frameless guide component of the IGS or FGS FRAMELESS GUIDING SYSTEM (STRYKER NAVIGATION, Kalamazoo, Mich.).

The retainer ring 611 can be made of any suitable material that is non-reactive in an MRI environment (e.g., MRI compatible polymers such as polyether ether ketone [PEEK] or polyetherimide [PEI; also known as Ultem®]). In accordance with various embodiments, the retainer ring 611 may contain a hole that can operatively engage with a latch or tongue on the frameless guide 610. In an exemplary embodiment, the retainer ring 611 contains a through-hole that allows the passage of a locking screw. The locking screw through-hole may be disposed on an opposite portion of the retainer ring 611 from the hole for the latch or tongue. The locking screw can engage with the threaded locking screw hole in the frameless guide 610 and can create firm but reversible contact between the retainer ring 611 and the frameless guide 610. The locking screw and latch hole enable quick but reliable positioning of the ball-stem adapter 612 at appropriate angles. In accordance with various embodiments, the retainer ring 611 may have one or more cutouts that reveal portions of the top surface of the frameless guide 610 when the two parts are mated.

The ball-stem adapter 612 can be made of any suitable material that is non-reactive in an MRI environment (e.g., MRI compatible polymers such as polyether ether ketone [PEEK] or polyetherimide [PEI; also known as Ultem®]). The ball-stem adapter 612 can help to properly set the trajectory of a surgical instrument with respect to a patient's skull 650. The ball-stem adapter may contain a through-hole that passes through the center of the adapter between the proximal end and the distal end. In various embodiments, the internal diameter of the through-hole of the ball-stem adapter 612 may be substantially constant throughout its entire length or may vary gradually or discretely along some segments of the length. The internal diameter of the through-hole may be chosen to be any size that satisfies application-specific requirements. In accordance with various embodiments, a distal portion 613 of the ball-stem adapter 612 may comprise a substantially spherical or ellipsoidal shape. The shape of the distal end 613 of the ball-stem adapter 612 may be mirrored in the shape of a through-hole in the frameless guide 610. A spherical or ellipsoidal shape of the distal end 613 of the ball-stem adapter 612 can enable the adapter to be rotated about its longitudinal axis to a position that is most convenient for use by the physician. In addition, the ball-stem adapter 612 may be tilted with respect to the normal defined by the frameless guide 610 to access trajectories that are away from the normal axis. In this way, the ball-stem adapter 612 maintains two degrees of rotational freedom, and multiple trajectories may be accessed using a single burr hole. In accordance with various embodiments, the proximal portion 614 of the ball-stem adapter 612 may comprise an end adapter that is configured to allow mounting of a surgical instrument.

In various embodiments, the frameless guide 610 and/or the ball-stem adapter 612 may include one or more fiducial markers 615. The fiducial markers 615 allow a user to register the position of the frameless guide 610 and/or the ball-stem adapter 612 in three-dimensional space over time and/or with previously-obtained imagery. The plurality of fiducial markers 615 may be imaged using one or more of a wide variety of imaging modalities including, but not limited to, magnetic resonance imaging, positron emission tomography, and/or x-ray imaging. In a preferred embodiment, the plurality of fiducial markers 615 are made from a non-paramagnetic material such as gold. The fiducial markers 615 may be attached to the frameless guide 610 and/or ball-stem adapter 612 using a variety of methods including, but not limited to, adhesive mounting, friction fit, retainer rings, welding, sintering, or vapor deposition. In accordance with various embodiments, the fiducial markers can be 2 mm spheres, and the distance between the markers can be about 10 mm. In various embodiments, the retainer ring 611 may have several cutouts that allow access to the fiducial markers 615 mounted on the frameless guide 610.

In accordance with various embodiments, a physician may use the plurality of fiducial markers 615 to register the location of the mounting system 600 and, hence, the patient's skull 650 during a surgical operation. The location of the fiducial markers 615 may be obtained using, for example, magnetic resonance imaging. As an example, the plurality of fiducial markers 615 can be identified on an MRI scan that is obtained in the MRI suite after the mounting device 602 has been secured to a patient's skull 650. The fiducial markers 615 can then be registered to the navigation system used in the operating room. With this registration, the location of the ball-stem adapter 612 on the skull 650 and the trajectory defined by the lumen of the ball-stem adapter 612 can be identified and shown on a navigation screen. The depth to a potential target can be measured, and the path chosen for the laser fiber can be examined for proximity to critical structures (such as blood vessels or anatomically delicate structures) before the fiber is advanced intracranially. With this approach, adjustments to the trajectory can be made before passing the laser fiber to avoid injury to critical structures. In addition, multiple trajectories can be utilized through a single opening by reorienting the trajectory of the ball-stem adapter 612 and re-registering to the navigation system. The fiducial markers 615 can provide advantages in terms of the time required to register the device and calculate a trajectory as compared to existing products. In addition, the ease and speed with which multiple trajectories can be calculated and executed are far superior to existing devices. In accordance with some embodiments, the total time (excluding scanning) required to calculate, evaluate and utilize a single planned trajectory, post intra-op MRI can be under thirty minutes whereas existing frame-based devices often require over 90 minutes to complete the same task. When setting up multiple trajectories, the additional time required with certain embodiments of this disclosure can be less than 20 minutes and would not require creation of a second entry point. Conversely, addressing multiple brain sites with a frame-based system can add an additional 45 minutes to one hour to the procedure.

By storing MRI images in a computer, the location of the fiducial markers 615 can be tracked over the time of the operation. In addition, the location of the fiducial markers 615 may be registered to previously acquired images of the patient's brain. In a preferred embodiment, the path from the exterior of the patient's skull 650 to the targeted, diseased portion of the brain is optimized using the location of the fiducial markers 615.

What is claimed is:

1. A neurosurgery system, comprising:
a frameless guide; and
a ball-stem adapter having a distal end that is adapted to attach to the frameless guide and having a proximal end that is adapted to be coupled to a laser ablation device.

2. The system of claim 1, further comprising a laser ablation device.

3. The system of claim 1, wherein the frameless guide comprises a base portion with a plurality of mounting holes and a plurality of surgical screws.

4. The system of claim 3, wherein the base portion is a flat plate.

5. The system of claim 3, wherein the base portion comprises a plurality of MRI-compatible fiducial markers.

6. The system of claim 5, wherein the ball-stem adapter includes a plurality of MRI-compatible fiducial markers.

7. The system of claim 6, wherein the plurality of MRI-compatible fiducial markers are made of a non-paramagnetic material.

8. The system of claim 7, wherein the non-paramagnetic material is gold.

9. The system of claim 6, wherein the minimum separation distance between each of the fiducial markers is at least ten millimeters.

10. The system of claim 1, wherein the ball-stem adapter is configured such that its position can be rotationally adjusted in two-degrees of freedom.

11. The system of claim 1, wherein the frameless guide further comprises a socket configured to receive the distal end of the ball-stem adapter.

12. The system of claim 1, wherein the distal end of the ball-stem adapter comprises a collar or flange having through holes.

13. A method of performing a surgical procedure on a skull or brain, comprising:
attaching a distal end of a ball-stem adapter to a frameless guide;
mounting the frameless guide to a skull of a patient;
coupling a surgical instrument to the proximal end of the ball-stem adapter; and
performing a surgical operation using the surgical instrument.

14. The method of claim 13, wherein the frameless guide comprises a base portion with a plurality of mounting holes and a plurality of surgical screws.

15. The method of claim 14, wherein the base portion is a flat plate.

16. The method of claim 14, wherein the base portion comprises a plurality of MRI-compatible fiducial markers.

17. The method of claim 16, wherein the ball-stem adapter includes a plurality of MRI-compatible fiducial markers.

18. The method of claim 17, wherein the plurality of MRI-compatible fiducial markers is made of a non-paramagnetic material.

19. The method of claim 18, wherein the non-paramagnetic material is gold.

20. The method of claim 17, wherein the minimum separation distance between each of the fiducial markers is at least ten millimeters.

21. The method of claim 13, wherein the ball-stem adapter is configured such that its position can be rotationally adjusted in two-degrees of freedom.

22. The method of claim 13, wherein the frameless guide comprises a socket to receive the distal end of the ball-stem adapter.

23. The method of claim 13, wherein the distal end of the ball-stem adapter is retained at the frameless guide by a retainer ring.

24. The method of claim 17, further comprising registering the spatial locations of the plurality of fiducial markers over time or with previously obtained images of a patient's brain.

25. The method of claim 24, further comprising determining an appropriate trajectory of the laser ablation probe to target an anatomical structure in the patient's brain based on a registered location of the plurality of fiducial markers.

26. The method of claim 13, wherein the step of mounting the frameless guide to a skull of a patient is performed proximal to a parietal lobe of the patient's brain.

27. The method of claim 13, wherein the step of mounting the frameless guide to a skull of a patient is performed proximal to an occipital lobe of the patient's brain.

28. The method of claim 13, wherein the surgical instrument is a laser ablation device and the surgical operation is a laser ablation operation.
